(19) United States
(12) Patent Application Publication
(10) Pub. No.: US 2006/0201342 A1
(43) Pub. Date: Sep. 14, 2006

(54) Use of porous metal membrane for treating liquid foods

(76) Inventors: Joseph E. Paganessi, Buit Ridge, IL (US); James T. C. Yuan, Naperville, IL (US)

Correspondence Address: Ms. Linda K. Russell;Air Liquide, Intellectual Property Dept., 2700 Post Oak Blvd., Suite 1800, Houston, TX, 77056, US

(21) Appl. No.: 11/356,456

(22) Filed: Feb. 16, 2006

Related U.S. Application Data
(60) Provisional application No. 60/657,677, filed on Mar. 1, 2005.

(51) Int. Cl. A23B 4/14

(52) U.S. Cl. 99/516

(57) ABSTRACT

Treatments for input fluids involve contacting a treatment fluid with the input fluid while maintaining substantial separation of the fluids. This contacting occurs in a reaction vessel having the treatment fluid disposed on one side of a porous metal membrane and the input fluid disposed on the other side of the porous metal membrane. Treatments can include disinfection and preservation of the input fluid and/or extraction of solutes from the input fluid.

### CROSS-REFERENCE TO RELATED APPLICATIONS

This application claims the benefit under 35 U.S.C. § 119(e) to provisional application No. 60/657,677, filed Mar. 1, 2005, the entire contents of which are incorporated herein by reference.

### BACKGROUND

Treatments for an input fluid can include a preservation process where microbes such as bacteria, viruses and/or spores in the input fluid are killed and/or enzymes which can catalyze undesired reactions in the input fluid are inactivated. Pasteurization offers the most commonly used process for disinfection and preservation of the input fluid. However, the pasteurization requires heating of the input fluid to temperatures which can degrade the quality of the input fluid. Heating of liquid foods for example can adversely affect the taste and nutritional quality of the food.

As an alternative to pasteurization, contacting the input fluid with pressurized dense carbon dioxide (CO₂) can disinfect and preserve the input fluid without requiring heating of the input fluid to damaging temperatures. Use of porous polymer membranes can allow this contact while desirably maintaining separation of the dense CO₂ and the input fluid. However, prior processes and equipment including those utilizing the polymer membranes for the contacting of the input fluid with the CO₂ present various problems and deficiencies.

For example, special precautions must be taken at extra expense and space to insure that a pressure differential does not rupture the polymer membranes. Other problems exist due to limited ability to in situ clean the polymer membranes which are susceptible to damage caused by high temperature and harsh chemicals associated with cleaning procedures. Further, plugging of the polymer membranes can occur due to suspended particulate matter in the input fluid not being able to pass through the limited internal diameters of the polymer membranes. In addition to these limitations, fluid treatment production rate may be undesirably low for some applications.

Other treatments for the input fluid can provide an extraction process that enables extraction of solutes from the input fluid. For treatments that provide this extraction by contacting a treatment fluid with the input fluid, the treatment fluids acting as a solvent can also advantageously be separated by a porous polymer membrane from the input fluid while still allowing contact between the fluids. Extraction of the solute from the treatment fluid enables recirculation of the treatment fluid, which would otherwise become saturated with the solute preventing further extraction. In practice, the extraction process can be substantially similar to the disinfection and preservation techniques since it can be necessary to establish saturation of the treatment fluid when the input fluid being preserved has a constituent that is soluble in the treatment fluid but is desired to not be extracted. Accordingly, the polymer membranes used in the extraction process can be substantially similar to those utilized for the disinfection and pasteurization procedures and thus are subject to the same types of problems.

Therefore, there exists a need for improved fluid treatment devices and processes that require both contact and substantial separation between two fluids.

### SUMMARY

In one embodiment, an apparatus for treating a fluid by exposure to a dense gas includes a reaction vessel having a porous metal membrane that defines a first flow path through the reaction vessel on a first side of the metal membrane and a second flow path through the reaction vessel on a second side of the metal membrane opposite the first side. The metal membrane substantially inhibits passage of the fluid through the metal membrane. An interaction of the dense gas and the fluid across the metal membrane, thereby effects a treatment of the fluid. The apparatus further includes a dense gas source coupled to a first pump for supplying the dense gas to the second flow path and a fluid source coupled to a second pump for supplying the fluid to the first flow path.

According to another embodiment, an apparatus for treating a liquid food by exposure to a gas includes a reaction vessel having a porous metal membrane that defines a first flow path through the reaction vessel on a first side of the metal membrane and a second flow path through the reaction vessel on a second side of the metal membrane opposite the first side. The metal membrane substantially inhibits passage of the liquid food through the metal membrane. The pores formed in the metal membrane are formed in a manner allowing contact between the gas and the liquid food via the pores while the gas and liquid food are flowing through their respective flow paths. The gas is selected to effect a desired treatment of the liquid food through the contact. Additionally, the apparatus includes a gas source coupled to a first pump for supplying the gas to the second flow path and a liquid food source coupled to a second pump for supplying the liquid food to the first flow path.

In a further embodiment, a method of treating a fluid by exposure to a gas includes providing a reaction vessel having a porous metal membrane disposed therein and flowing the fluid along a first flow path through the reaction vessel. The first flow path is separated from a second flow path through the reaction vessel by the metal membrane. The method additionally includes flowing the gas along the second flow path, wherein the respective pressures of the fluid and the gas create a pressure differential across the metal membrane, thereby causing contact between the fluid and the gas via pores formed in the metal membrane during the treating. Contacting the fluid and gas via the pores effects a desired treatment of the fluid.

For yet another embodiment, a method of treating a fluid by exposure to a dense gas includes providing a reaction vessel having a porous metal membrane disposed therein and flowing the fluid along a first flow path through the reaction vessel. The first flow path is separated from a second flow path through the reaction vessel by the metal membrane. Furthermore, the method includes flowing the dense gas along the second flow path thereby contacting the fluid and dense gas at pores in the metal membrane and cleaning the porous metal membrane in place within the reaction vessel.

### BRIEF DESCRIPTION OF THE DRAWINGS

For a further understanding of the nature and objects of the invention, reference should be made to the following detailed description, taken in conjunction with the accompanying drawings, in which like elements are given the same or analogous reference numbers and wherein:

FIG. 1 illustrates a schematic of a system for sanitizing an input fluid, according to embodiments of the invention;

FIG. 2 illustrates a cut away perspective view of a reaction vessel having a porous metal membrane conduit disposed in an enclosure, according to embodiments of the invention;

FIG. 3 illustrates a schematic of another system for sanitizing an input fluid, according to embodiments of the invention;

FIG. 4 illustrates a schematic of a system for extracting a solute from an input fluid, according to embodiments of the invention; and

FIG. 5 illustrates a schematic of one application for the system such as shown in FIG. 1, according to embodiments of the invention.

### DESCRIPTION OF PREFERRED EMBODIMENTS

Embodiments of the invention generally relate to methods and apparatus for treating fluids. Fluid treatment devices and processes disclosed herein rely on both contact and at least some separation between a treatment fluid and an input fluid to be treated. Contacting the treatment fluid with the input fluid occurs in a reaction vessel where the treatment fluid is disposed on one side of a porous metal membrane and the input fluid is disposed on the other side of the porous metal membrane. Treatments can include disinfection and preservation of the input fluid and/or extraction of solutes from the input fluid. Some embodiments incorporate clean-in-place or in situ apparatus cleaning procedures. Further, improved production rates for treating the input fluid can be achieved due to aspects of the invention.

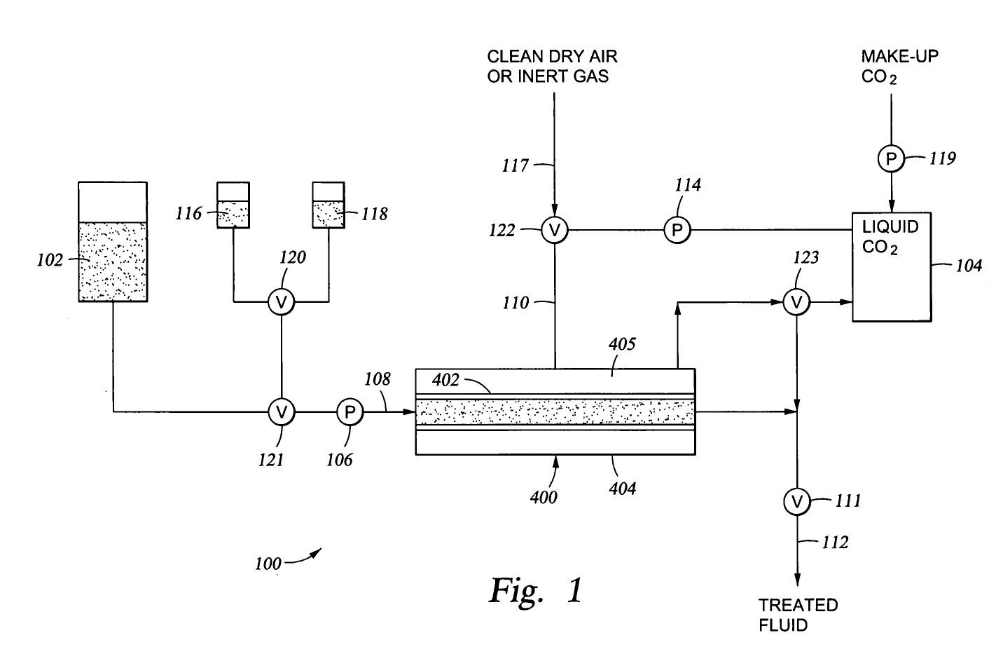

FIG. 1 shows a system 100 for sanitizing an input fluid 102. The input fluid 102 can be any aqueous liquid that can be disinfected by being contacted with an appropriate treatment fluid 104. For example, the input fluid 102 can include beverages, fruit juice, fruit puree, vegetable juice, vegetable puree or liquid medicines. Illustratively, FIG. 1 shows the treatment fluid 104 as dense carbon dioxide (CO₂), but other fluids are contemplated including nitrous oxide (N₂O), hydrogen (H₂), oxygen (O₂), nitrogen (N₂), argon (Ar), nitrogen monoxide (NO) and mixtures thereof.

The system 100 includes a reaction vessel 400 in fluid communication with an input fluid inlet 108 and an output 112 for discharging the input fluid 102 after being treated within the reaction vessel 400. A feed pump 106 pressurizes the input fluid 102 prior to delivery to an interior of a metal membrane conduit 402 via the inlet 108. The reaction vessel 400 provides part of a circulation path 110 of the dense CO₂ 104, which can be introduced/replenished by supply of make-up CO₂ from make-up pump 119. Flow of the dense CO₂ 104 continually circulates through the flow path 110 by a recirculation pump 114. The circulation path 110 enables flow of the dense CO₂ 104 in an annulus 405 between an external surface of the metal membrane conduit 402 and an inside surface of an enclosure 404 surrounding the metal membrane conduit 402. The metal membrane conduit 402 maintains substantial separation of the input fluid 102 from the circulation path 110 of the dense CO₂ 104, while allowing some controlled interaction (contact) between the input fluid 102 with the dense CO₂ 104. One embodiment of the reaction vessel 400 will be described below. After treatment of the input fluid 102 in the reaction vessel 400, output valve 111 regulates flow from the system 100 of treated fluid through the output 112.

The system 100 further includes, for some embodiments, a supply for cleaning fluid 116 in fluid communication with the inlet 108. The cleaning fluid 116 can enable in situ cleaning of the system 100 including the reaction vessel 400 and the metal membrane conduit 402 disposed therein. For some embodiments, always maintaining a positive pressure in the circulation path 110 relative to fluid pressure at the inlet 108 prevents flow of unprocessed fluid product into the circulation path 110 of the dense CO₂ 104 thereby keeping the circulation path 110 free from contamination. This positive pressure can be accomplished during cleaning by using CO₂ gas or compressed clean air 117 when the cleaning fluid 116 being used chemical reacts with CO₂.

Cleaning of the system 100 can occur before/after use of the system 100 or at scheduled or on demand times. At these occasions, valves 120-123 selectively divert flow of the cleaning fluid 116 and optionally the clean air 117 through the system. The cleaning fluid 116 can exit the system 100 through a separate drain or be passed through the output 112 while there is no collection being made of the treated fluid. For some embodiments, cleaning fluid can be introduced manually into, for example, the inlet 108 during servicing thereby still permitting in situ cleaning of the metal membrane conduit 402.

The cleaning fluid 116 can include heated water or chemical agents that dissolve and/or kill contaminants without dissolving or attacking the metal membrane conduit 402. Examples of chemical agents for use as the cleaning fluid 116 include 15% nitric acid at up to about 65° C., 20% hydroxide of a light metal at up to about 100° C., alcohols, acetic acid, acetone, ammonia, organic solvents, methylene chloride, and other solvents detergents and industrial cleaners. Optionally, a rinse fluid 118 in fluid communication with the inlet 108 enables rinsing of the system 100 following passing of the cleaning fluid 116 through the system 100.

For some embodiments, a cleaning procedure involves introducing the cleaning fluid 116 into the reaction vessel 400 and allowing the metal membrane conduit 402 to soak in the selected chemical agent within the cleaning fluid 116. Thereafter, flushing the reaction vessel 400 with the rinse fluid 118 removes the cleaning fluid 116 and debris from the system 100. The rinse fluid 118 can be any combination of clean, filtered water, clean air and steam. Where two chemical agents are required to perform the cleaning, a water flush with the rinse fluid 118 can be performed between soaks.

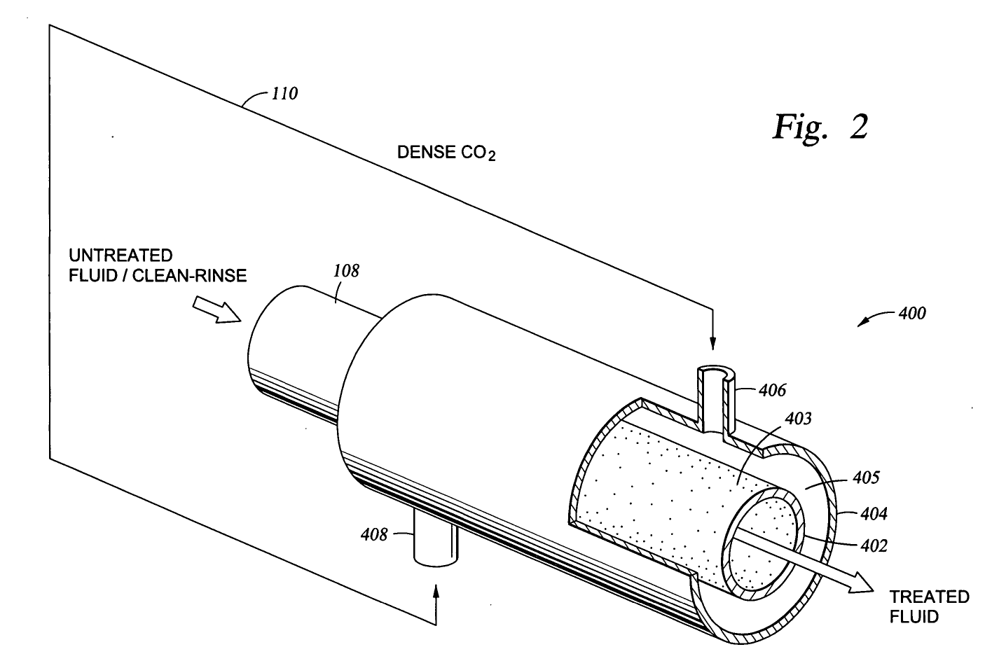

Referring now to FIG. 2, a cross-sectional perspective view of the reaction vessel 400 is shown. Illustratively, the reaction vessel 400 has a circular cross section; however, it is contemplated that the cross-section may be elliptical or of any shape. In general, the metal membrane conduit 402 is concentrically disposed within the enclosure 404. In this arrangement, the metal membrane conduit 402 defines a fluid flow path for the input fluid 102, while the outer surface of the metal membrane conduit 402 and the inner surface of the enclosure 404 define the annulus 405 through which the treatment fluid is flowed. Inlet and outlet ports 408, 406 are formed in the enclosure 404 and define the locations of the vessel 400 at which the CO₂ 104 is input to the annulus 405 and where the CO₂ 104 is removed from the annulus 405. Illustratively, the inlet and outlet ports 408, 406 are located on opposite sides (e.g., top and bottom) of the enclosure 404 and proximate each end of the metal membrane conduit 402. In this configuration, the flow path 110 travels along and around the length of the metal membrane conduit 402.

As noted above, separation of the input fluid 102 and the CO₂ 104 is substantially maintained while the respective fluids are flowing through the reaction vessel 400. However, contact of the input fluid 102 with the dense CO₂ 104 occurs at minute pores 403 which penetrate through the metal membrane conduit 402.

The metal membrane conduit 402 inhibits passage of the input fluid 102 through the pores 403 of the metal membrane conduit 402 since the metal membrane conduit 402 is not wetted by the input fluid 102 and the size of the pores 403 is limited. The size of the pores 403 of the metal membrane conduit 402, for some embodiments, is 0.2 micron. Diameters of the pores 403 can range from about 0.001 micron to about 1.0 micron if the wall thickness of the metal membrane conduit 402 is within the range from about 0.005 mm to about 3 mm. Selection of the largest size for the pores 403 that inhibits passage of the input fluid 102 through the metal membrane conduit 402 maximizes the interface area for the input fluid 102 and the dense CO₂ 104. Additionally, increasing porosity of the metal membrane conduit 402 can raise processing rates.

According to one embodiment, it is contemplated that the dense CO₂ 104 and the input fluid 102 are maintained in pressurized states while flowing through the reaction vessel 400. Pressurization of both the dense CO₂ 104 outside of the metal membrane conduit 402 and the input fluid 102 within the metal membrane conduit 402 can be from about 1000 pounds per square inch (psi) to about 3000 psi, and operating temperature can be from about −10° C. to about 400° C. For some embodiments, pressures of the dense CO₂ 104 and the input fluid 102 are maintained at different pressures during treatment within the reaction vessel 400 in order to improve interaction contact between the input fluid 102 and the dense CO₂ 104. Regardless of whether these different pressures are intended or occur incidentally, the metal membrane conduit 402 provides sufficient structural integrity to avoid collapsing or rupturing at desired or potential differential pressures.

For some embodiments, pressure of the input fluid 102 can be maintained higher or lower than the pressure of the dense CO₂ 104 to provide a differential pressure. This differential pressure creates a pressure gradient along with a concentration gradient across the metal membrane conduit 402 thereby enhancing the interaction contact. As a result of this improved contact, the metal membrane conduit 402 achieves adequate contact between the input fluid 102 and the dense CO₂ 104 with relatively smaller dispersion areas for the input fluid 102 and the dense CO₂ 104 interaction. It is contemplated that the densities of the input fluid 102 and the dense CO₂ 104 are the same or different. Furthermore, operating the system 100 with the differential pressure results in reducing or eliminating undesired dissolution or extraction of volatile organic chemicals from the input fluid 102 into the treatment fluid such as the dense CO₂ 104.

When the pressure of the input fluid 102 is higher than the pressure of the dense CO₂ 104, the differential pressure can be less than 10.0 psi, or less than 15.0 psi, based on wetability of the metal membrane conduit 402 by the input fluid 102, which tends to force the input fluid 102 into the dense CO₂ 104. Alternatively, the differential pressure can be less than 2.0 psi, or less than 5.0 psi, based on wetability of the metal membrane conduit 402 by the dense CO₂ 104, which tends to force the dense CO₂ 104 into the input fluid 102 when the pressure of the dense CO₂ 104 is higher than the pressure of the input fluid 102. In either case, the pressure differential for some embodiments is greater than 1.0 psi.

In addition to making possible the advantageous differential pressure, the structural integrity of the metal membrane conduit 402 enables utilization of an internal diameter for the metal membrane conduit 402 that is relatively large. The internal diameter of the metal membrane conduit 402 being large allows for high flow rates and large particle sizes of non-liquid suspensions. For some embodiments, the internal diameter of the metal membrane conduit 402 is greater than 0.6 millimeters (mm). Additionally, the inner diameter of the metal membrane conduit 402 being large reduces or eliminates the potential for blockage. This blockage can stop flow of the input fluid 102 through the reaction vessel 400 altogether or lead to dead space within the reaction vessel 400, which adds to the potential for contamination. Diameter of the metal membrane conduit 402 can be selected as a function of the flow rate of fluid and size of particles in the fluid being processed.

Based on the foregoing criteria for the metal membrane conduit 402, fabrication of the metal membrane conduit 402 can include any processes that provide an interconnected network of the pores 403 bounded by bonded metal. For example, the metal membrane conduit 402 can be molded from a controlled mixture of metal powder and sacrificial pore-formers that are compacted and subsequently sintered in furnaces. Controlling the chemistry and size of the pore-formers as well as the shape, size and distribution of the metal powders provides a fixed porous network upon sintering, which meets the criteria for the metal membrane conduit 402. Further, the metal membrane conduit 402 can be manufactured in a wide variety of materials including stainless steel alloys, nickel, titanium, Monel®, Inconel® and Hastelloy®.

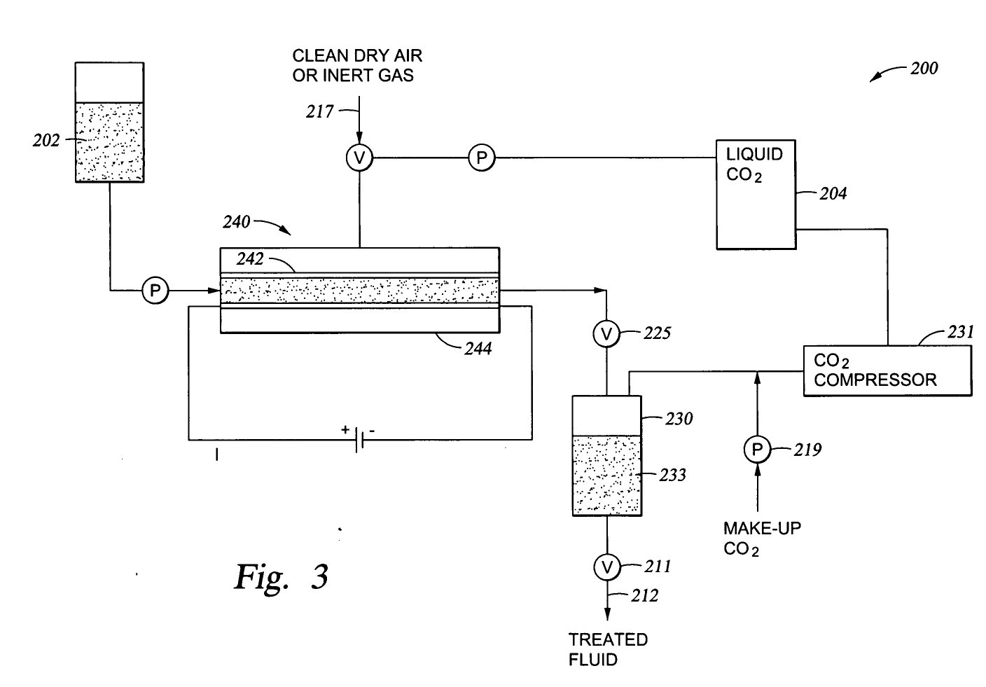

FIG. 3 illustrates a system 200 for sanitizing an input fluid 202 by utilizing a reaction vessel 240 to contact the input fluid 202 with a dense CO₂ 204. The reaction vessel 240 includes a metal membrane conduit 242 in accordance with aspects of the invention previously described. In addition, the system 200 includes a pressure reducer valve 225, a separator 230, and a compressor 231. The separator 230 can be a low pressure tank maintained at atmospheric pressure. In operation, the input fluid 202 can acquire some of the dense CO₂ 204 while passing through the reaction vessel 240 such that thereafter the dense CO₂ 204 separates out of a liquid 233 within the separator 230. The compressor 231 intakes from a top region of the separator 230 and liquefies the dense CO₂ 204 for recirculation. Additionally, the compressor 231 can receive make-up CO₂ from make-up pump 219. Valve 211 regulates flow from the system 200 of treated fluid that drains through the output 112 from a bottom portion of the separator 230.

FIG. 3 also shows an alternative in situ cleaning arrangement for the system 200 instead of using cleaning fluid in situ as shown in FIG. 1. A current (I) is passed along the reaction vessel 240 through any combination of the metal membrane conduit 242, a surrounding enclosure 244 or a separate heating element. Resistances to this current flow can thus either directly or through conduction raise the temperature of at least a region along the metal membrane conduit 242 to a level required for disinfection. Prior to cleaning at high temperatures, the input fluid 202 can be shut off and the reaction vessel 240 evacuated with the dense CO₂ 204 or a supply of clean dry air or inert gas 217.

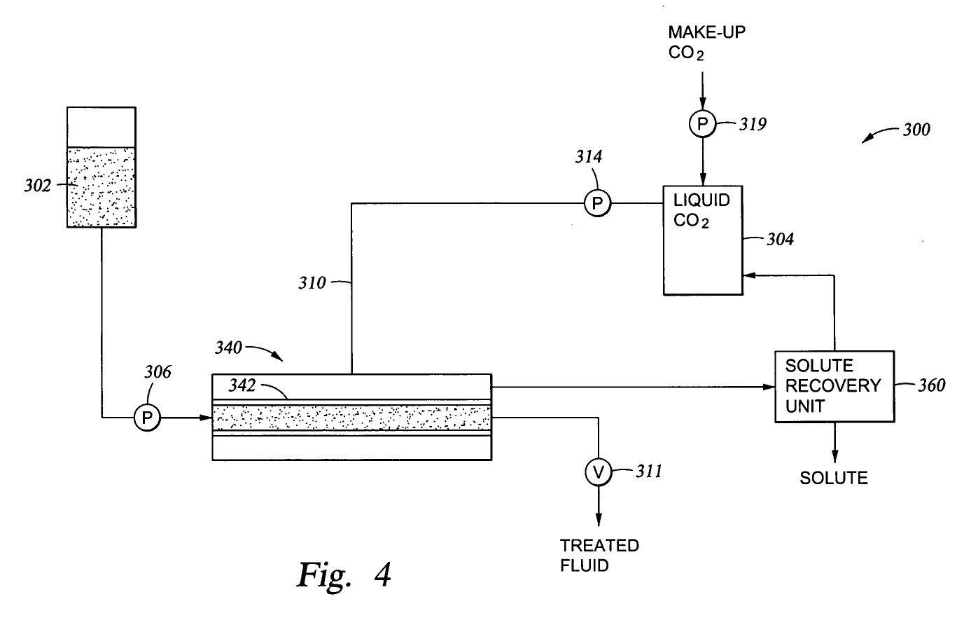

FIG. 4 illustrates a system 300 for extracting a solute from an input fluid 302. The solute to be extracted can be any solute with some solubility in both the input fluid 302 and a selected treatment fluid. For example, the solute can include a flavor, fragrance, caffeine, or a pharmaceutical or chelated metal. Similar to input fluids associated with preservation techniques heretofore discussed, the input fluid 302 can include beverages, fruit juice, fruit puree, vegetable juice, vegetable puree, liquid medicines, oil-in-water emulsions, live cell fermentor broth and enzyme broth. While the system 300 is shown utilizing a dense CO₂ 304 as the treatment fluid for extraction of the solute, further suitable treatment fluids include other dense gases such as N₂O, methane, ethane, propane, butane, isobutane, ethene, propene, tetrafluoromethane, chlorodifluoromethane, sulphur hexaflouride, ammonia, methyl chloride and hydrofluorocarbons, which include partially fluorinated methanes, ethanes and propanes, such as fluromethane, trifluoromethane, tetrafluoroethane, 1,1,1,2,3,3,3-heptafluoropropane, and mixtures thereof. The dense CO₂ 304 can be introduced/replenished by supplying make-up CO₂ from make-up pump 319.

By contrast, FIGS. 1 and 3 show embodiments suited for fluid preservation without extraction since even if the input fluids 102, 202 contain a solute soluble in the dense CO₂ 104, 204 saturation of the dense CO₂ 104, 204 with the solute occurs and prevents removal of any such constituents. Thus, addition of a solute recovery unit 360 within a circulation flow path 310 for the dense CO₂ 304 operates to remove the solute. Other than this addition of the solute recovery unit 360, the system 300 includes pumps 314, 306, a reaction vessel 340, and a valve 311, which function in the same manner for contacting the dense CO₂ 304 with the input fluid 302 under such conditions as sanitizing procedures described above with reference to FIGS. 1-3 and corresponding elements.

Therefore, a metal membrane conduit 342 within the reaction vessel 340 provides all of the benefits described herein to the system 300. Additionally, cleaning procedures can be incorporated for use with the system 300. The metal membrane conduit 342 can be cleaned with any combination of high temperature and cleaning fluids or chemicals such as caustics.

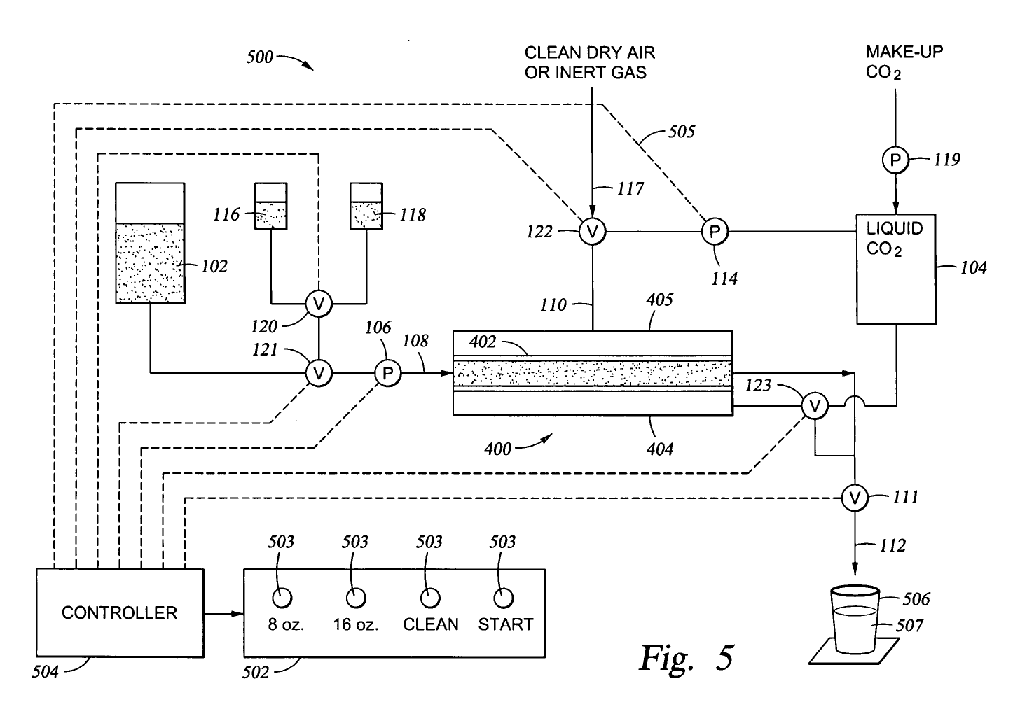

FIG. 5 illustrates a juice dispensing machine 500 that incorporates the system shown in FIG. 1. Accordingly, the same reference numbers have been used to identify the identical components in these figures. Examples of the juice dispensing machine 500 include vending machines and counter top dispensers such as provided at convenience and grocery stores, restaurants and hotels. With the machine 500, a customer can collect treated juice 507 in a cup 506 positioned below the output 112. The treated juice 507 has been disinfected upon passing through the reaction vessel 400.

The machine 500 includes a controller 504 and an interface such as a touch pad 502. The touch pad 502 includes buttons 503 for making selections. The controller 504 receives inputs from the touch pad 502 and sends signals along lines 505 to control actuation of appropriate ones of the valves and pumps 120-123, 111, 106, 114. In operation of the machine 500, the customer pushes appropriate ones of the buttons 503 to select the size/type of juice and start dispensing of the treated juice 507.

Preferred processes and apparatus for practicing embodiments of the invention have been described. It will be understood and readily apparent to the skilled artisan that many changes and modifications may be made to the above-described embodiments without departing from the spirit and the scope of the invention. For example, features shown and described with one system may be interchanged or combined with those of other systems. The foregoing is illustrative only and that other embodiments of the integrated processes and apparatus may be employed without departing from the true scope of the invention defined in the following claims.

What is claimed is:

1. An apparatus for treating a fluid by exposure to a dense gas, comprising:
a reaction vessel having a porous metal membrane that defines a first flow path through the reaction vessel on a first side of the metal membrane and a second flow path through the reaction vessel on a second side of the metal membrane opposite the first side, wherein the metal membrane substantially inhibits passage of the fluid through the metal membrane and wherein an interaction of the dense gas and the fluid across the metal membrane, thereby effects a treatment of the fluid;
a dense gas source coupled to a first pump for supplying the dense gas to the second flow path; and
a fluid source coupled to a second pump for supplying the fluid to the first flow path.

2. The apparatus of claim 1, wherein the metal membrane is formed of a sintered mixture of metal powder and sacrificial pore-formers.

3. The apparatus of claim 1, wherein the first pump is configured to supply the dense gas at a first pressure and the second pump is configured to supply the fluid at a second pressure different from the first pressure.

4. The apparatus of claim 1, wherein the first pump is configured to supply the dense gas at a first pressure and the second pump is configured to supply the fluid at a second pressure, the first and second pressure defining a pressure differential that is at least 1.0 pounds per square inch.

5. The apparatus of claim 1, wherein the fluid source comprises a liquid food.

6. The apparatus of claim 1, wherein the fluid source comprises a fruit juice.

7. The apparatus of claim 1, wherein the dense gas source supplies the dense gas comprising carbon dioxide.

8. The apparatus of claim 1, wherein the dense gas source supplies the dense gas comprising at least one of nitrous oxide (N₂O), hydrogen (H₂), oxygen (O₂), nitrogen (N₂), argon (Ar) and nitrogen monoxide (NO).

9. The apparatus of claim 1, further comprising a cleaning fluid source in fluid communication with the reaction vessel for supplying a cleaning fluid to the metal membrane.

10. The apparatus of claim 1, further comprising a cleaning fluid source in fluid communication with the reaction vessel for supplying a caustic fluid to the metal membrane.

11. The apparatus of claim 1, further comprising a solute recovery unit configured to collect solute extracted from the fluid.

12. The apparatus of claim 1, further comprising:
a user interface for customer selection; and
a controller configured to receive inputs from the user interface and based on the inputs control operations to dispense the customer selection obtained by treatment of the fluid.

13. The apparatus of claim 1, further comprising a heating assembly configured to provide thermal energy to a region along the metal membrane during cleaning operations.

14. An apparatus for treating a liquid food by exposure to a gas, comprising:
a reaction vessel having a porous metal membrane that defines a first flow path through the reaction vessel on a first side of the metal membrane and a second flow path through the reaction vessel on a second side of the metal membrane opposite the first side, wherein the metal membrane substantially inhibits passage of the liquid food through the metal membrane, and wherein pores formed in the metal membrane are formed in a manner allowing contact between the gas and the liquid food via the pores while the gas and liquid food are flowing through their respective flow paths, and wherein the gas is selected to effect a desired treatment of the liquid food through the contact;
a gas source coupled to a first pump for supplying the gas to the second flow path; and
a liquid food source coupled to a second pump for supplying the liquid food to the first flow path.

15. The apparatus of claim 14, wherein the metal membrane is formed of a sintered mixture of metal powder and sacrificial pore-formers.

16. The apparatus of claim 14, wherein the metal membrane is a tubular member defining the first flow path along its central axis.

17. The apparatus of claim 16, wherein the metal membrane is concentrically disposed in an enclosure and wherein the second flow path is defined by an annulus formed between an inner surface of the enclosure and an outer surface of the metal membrane.

18. The apparatus of claim 14, wherein the desired treatment of the fluid is one of disinfection of the liquid food and preservation of the liquid food.

19. A method of treating a fluid by exposure to a gas, comprising:
providing a reaction vessel having a porous metal membrane disposed therein;
flowing the fluid along a first flow path through the reaction vessel, wherein the first flow path is separated from a second flow path through the reaction vessel by the metal membrane; and
flowing the gas along the second flow path, wherein the respective pressures of the fluid and the gas create a pressure differential across the metal membrane, thereby causing contact between the fluid and the gas via pores formed in the metal membrane during the treating; and wherein contacting the fluid and gas via the pores effects a desired treatment of the fluid.

20. The method of claim 19, wherein the desired treatment of the fluid is one of disinfection of the fluid, preservation of the fluid, and extraction of solutes from the fluid.

21. The method of claim 19, wherein the metal membrane is a tubular member defining the first flow path along its central axis.

22. The method of claim 21, wherein the metal membrane is concentrically disposed in an enclosure and wherein the second flow path is defined by an annulus formed between an inner surface of the enclosure and an outer surface of the metal membrane.

23. The method of claim 19, wherein the pressure differential is at least 1.0 pounds per square inch.

24. The method of claim 19, further comprising dispensing a treated beverage obtained from the fluid after being passed along the first flow path.

25. The method of claim 19, wherein the dense gas comprises carbon dioxide.

26. The method of claim 19, further comprising passing a cleaning fluid across the metal membrane.

27. The method of claim 19, further comprising passing a caustic across the metal membrane.

28. A method of treating a fluid by exposure to a dense gas, comprising:
providing a reaction vessel having a porous metal membrane disposed therein;
flowing the fluid along a first flow path through the reaction vessel, wherein the first flow path is separated from a second flow path through the reaction vessel by the metal membrane;
flowing the dense gas along the second flow path thereby contacting the fluid and dense gas at pores in the metal membrane; and
cleaning the porous metal membrane in place within the reaction vessel.

29. The method of claim 28, wherein cleaning the porous metal membrane includes flowing a caustic fluid across the metal membrane.

30. The method of claim 28, wherein cleaning the porous metal membrane includes increasing the temperature in a region along the metal membrane.
